(19) United States
(12) Patent Application Publication
(10) Pub. No.: US 2022/0323339 A1
(43) Pub. Date: Oct. 13, 2022

(54) CLEANSER

(71) Applicant: baresop Pty Ltd, Elwood (AU)

(72) Inventors: Prisca Ongonga-Daehn, Elwood (AU); Saul Pyle, Lilyfield (AU)

(73) Assignee: baresop Pty Ltd, Elwood (AU)

(21) Appl. No.: 17/544,672

(22) Filed: Dec. 7, 2021

(30) Foreign Application Priority Data
Apr. 1, 2021 (AU) 2021202045

(51) Int. Cl. A61K 8/92; A61Q 19/10; A61Q 19/00; A61K 8/04; A61K 8/46; A61K 8/34; A61K 8/368; A61K 8/365; A61K 8/49; A61K 8/37; A61K 8/25

(52) U.S. Cl. CPC A61K 8/922; A61Q 19/10; A61Q 19/007; A61K 8/046; A61K 8/466; A61K 8/345; A61K 8/368; A61K 8/365; A61K 8/4913; A61K 8/375; A61K 8/25

(57) ABSTRACT

The present invention provides a solid foaming composition. The composition comprises a solid surfactant, a chelator, a preservative, a pH buffer, an anticaking agent and an emollient.

### FIELD OF THE INVENTION

The present invention relates to a solid product or a concentrate that can be used to produce a cleansing product and in particular to produce a hand cleanser or body wash.

The invention has been developed primarily for use in personal cleansers and will be described hereinafter with reference to this application. However, it will be appreciated that the invention is not limited to this particular field of use.

### BACKGROUND OF THE INVENTION

The average household in Australia uses around 30 bottles of handsoap per year.

Most hand soap and body wash products comprise around 90% water, with only about 10% of these products being active materials. The presence of such a large proportion of water increases the need for packaging and increases the cost and carbon footprint associated with transporting the products.

There is therefore a need for a product with a substantially lower water content which can be used to produce hand soap and/or body wash.

The present invention seeks to provide a low water content product, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

According to a first aspect, there is provided a solid foaming composition comprising, optionally consisting essentially of, a solid surfactant, a chelator, a preservative, a pH buffer, an anticaking agent and an emollient.

The following options may be used in conjunction with the first aspect, either individually or in any suitable combination.

The surfactant may be present at about 50 to about 90% of the composition. The chelator may be present at about 1 to about 5% of the composition. The preservative may be present at about 5 to about 20% of the composition. The pH buffer may be present at about 5 to about 10% of the composition. The anticaking agent may be present at about 1 to about 5% of the composition. The emollient may be present at about 1 to about 5% of the composition.

The composition may additionally comprise a solid skin conditioner. In this event, the skin conditioner may be present at about 10 to about 25% of the composition.

The composition may additionally comprise an essential oil. In this event, the composition may additionally comprise a compatibiliser. The compatibiliser is a composition which can serve to compatibilise the essential oil with an aqueous environment. If the composition comprises an essential oil, the essential oil may be present at about 0.1 to about 0.5% of the composition. In the event that the composition additionally comprises a compatibiliser, the compatibiliser may be present at from 5 to about 10 times the weight of the essential oil. In some instances, the essential oil may be substituted by, or complemented by, a different fragrance substance.

The composition may comprise less than about 0.5% free water.

In an embodiment there is provided a solid foaming composition comprising a solid surfactant at about 50-90%, a chelator at about 1-5%, a preservative at about 5-20%, a pH buffer at about 5-10%, an anticaking agent at about 1-5% and an emollient at about 1-5%. These amounts may sum to about 98%, or to about 99 or 100%.

In a further embodiment there is provided a solid foaming composition comprising, optionally consisting essentially of, a solid surfactant, a solid skin conditioner, a chelator, a preservative, a pH buffer, an anticaking agent, an essential oil, a compatibiliser and an emollient.

In a yet a further embodiment there is provided a solid foaming composition comprising, optionally consisting essentially of, a solid surfactant at about 50-90% or about 50-60%, a solid skin conditioner at about 10-25%, a chelator at about 1-5%, a preservative at about 5-20%, a pH buffer at about 5-10%, an anticaking agent at about 1-5%, an essential oil at about 0.1-0.5%, a compatibiliser at about 0.5-5% and an emollient at about 1-5%. These amounts may sum to about 98%, or to about 99 or 100%.

In still a further embodiment there is provided a solid foaming composition comprising a solid surfactant at about 54%, a solid skin conditioner at about 20%, a chelator at about 2.5%, a preservative at about 10%, a pH buffer at about 7.5%, an anticaking agent at about 2%, an essential oil at about 0.2%, a compatibiliser at about 2% and an emollient at about 2%.

It should be understood that each of the components of the composition mentioned above may be a single substance or may be a blend of substances. Thus for example where it is stated that there may be a preservative at about 5-20%, there may be more than one preservative with a total amount of 5-20%. Similarly, where there is mention of a pH buffer at about 5-10%, this may comprise an acid together with its conjugate base, with a total amount of about 5-10%, or may even comprise more than one acid together with their respective conjugate bases, at a total amount of about 5-10%.

In a second aspect of the invention there is provided a process for making a solid foaming composition comprising blending a solid surfactant, a chelator, a preservative, a pH buffer, an anticaking agent and an emollient.

The following options may be used in conjunction with the second aspect, either individually or in any suitable combination.

The process may comprise milling the solid surfactant prior to said blending. It may comprise sieving the solid surfactant after said milling.

The process may comprise Step A: combining the chelator, the buffer and the preservative to produce a first blend, and Step B: blending the first blend with the solid surfactant to form a second blend. It should be noted that the terms “blend” (as a noun) and “mixture” are used interchangeably and take the same meaning as each other.

The process may comprise Step C: combining the second blend with at least a portion of the anticaking agent to form a third blend. Step C may comprise combining an essential oil and a compatibiliser to form an oil-compatibiliser mixture and adding the oil-compatibiliser mixture to the anticaking agent under agitation, so as to form a fourth blend, and combining the second blend and the fourth blend so as to form the third blend. In some embodiments, step C is not conducted.

The process may comprise Step D: combining the emollient with the third blend so as to form the composition. Step D may comprise combining the emollient with a portion of the anticaking agent to form a fifth blend and adding the fifth blend to the third blend so as to form the composition. The fifth blend may be a clear liquid.

Step A may comprise combining the chelator, the buffer, the preservative and a solid skin conditioner so as to produce the first blend.

In an embodiment there is provided a process for making a solid foaming composition comprising:

Step A: combining a chelator, a buffer and a preservative to produce a first blend;
Step B: blending the first blend with a solid surfactant to form a second blend Optionally Step C: combining the second blend with at least a portion of an anticaking agent to form a third blend; and Step D: combining an emollient with the third blend so as to form the composition.

In another embodiment there is provided a process for making a solid foaming composition comprising:

Step A: combining a chelator, a pH buffer, a preservative and a solid skin conditioner so as to produce a first blend;
Step B: blending the first blend with a solid surfactant to form a second blend;
Step C: combining an essential oil and a compatibiliser to form an oil-compatibiliser mixture and adding the oil-compatibiliser mixture to an anticaking agent under agitation, so as to form a fourth blend, and combining the second blend and the fourth blend so as to form a third blend; and
Step D: combining an emollient with further of the anticaking agent to form a fifth blend and adding the fifth blend to the third blend so as to form the composition.

In yet another embodiment there is provided a process for making a solid foaming composition comprising:

Step A: combining about 1-5 parts of a chelator, a about 5-10 parts of pH buffer, about 5-20 parts of a preservative and about 10-25 parts of a solid skin conditioner so as to produce a first blend;
Step B: blending the first blend with about 50-70 parts of a solid surfactant to form a second blend;
Step C: combining about 0.1-0.5 parts of an essential oil and about 0.5-5 parts of a compatibiliser to form an oil-compatibiliser mixture and adding the oil-compatibiliser mixture to about 0.5-2 parts of an anticaking agent under agitation, so as to form a fourth blend, and combining the second blend and the fourth blend so as to form a third blend; and
Step D: combining about 1-5 parts of an emollient with about 0.5-2 parts of the anticaking agent to form a fifth blend and adding the fifth blend to the third blend so as to form the composition;
wherein all parts are by weight and the total of the parts adds to about 100 parts.

In a third aspect of the invention there is provided a process for making a liquid foaming composition, said process comprising combining the composition of the first aspect with water and agitating the resulting mixture. The agitating may be for a time of between about 10 and 60 seconds. The combining may be in a ratio of 1 part by weight of said composition to from 20 to 50 parts by weight of water. The water may be at a temperature of between about 30 and about 90° C.

The liquid foaming composition may be a sanitiser, shampoo, a hair conditioner, a face cleanser, a handwash or a body wash.

In an embodiment, there is provided a process for making a liquid foaming composition, said process comprising combining the composition of the first aspect with water and agitating the resulting mixture, wherein the agitating is for a time of between about 10 and 60 seconds, the combining is in a ratio of 1 part by weight of said composition to from 20 to 50 parts by weight of water and the water is at a temperature of between about 30 and about 90° C.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

In the present specification, all percentages are on a w/w basis unless specified otherwise.

### DETAILED DESCRIPTION

The present invention may be illustrated with reference to a solid foaming composition which may be dissolved and/or suspended and/or dispersed in water to produce a liquid soap. This will be described below. The term “solid foaming composition” refers herein to a solid composition which, when diluted with water, provides a liquid which, on agitation can generate a foam. A solid may be a monolithic solid or it may be a powder or it may be a granular solid or it may be a paste. In particular, it may be a free-flowing powder. It may be readily soluble in water. It may be readily soluble in water at about 30 to about 90° C. It will be recognised that certain components of the composition, for example the anticaking agent, may not be water soluble. In that instance, these components may be readily dispersible in water.

An important aspect of a soap product is a solid surfactant. This provides the cleansing properties of the composition. The surfactant may be a foaming surfactant. It may be biodegradable. It may have an HLB of greater than 10, or greater than 11, 12, 13, 14 or 15, or may have an HLB of about 10 to 18, 10 to 16, 12 to 18, 12 to 16 or 13-15, e.g. about 10, 11, 12, 13, 14, 15, 16, 17 or 18. It may be an anionic surfactant. It may be a sodium salt. It may be a sodium alkanoyl methyl isethionate, e.g. sodium lauroyl methyl isethionate. A suitable supplier of this material is Innospec Inc., who sells it under the name Iselux®. The solid surfactant may be present in the composition at about 50 to about 90% by weight, or about 50 to 70, 70 to 90, 50 to 60, 60 to 70, 70 to 80 or 80 to 90%, e.g. about 50, 55, 60, 65, 70, 75, 80, 85 or 90%. It should be solid at temperatures up to about 100° C., or up to, or at, about 90, 80, 70, 60, 50 or 40° C. The solid surfactant may be a mixture of solid surfactants as described above. It may be a mixture of 2, 3, 4 or 5 surfactants. In the event that a mixture of surfactants is used, the combined amounts of the solid surfactants may be as described above (i.e. about 50-90%).

Another component of the solid composition is a chelator. This may ensure that the composition may be readily diluted even if hard water or water containing unwanted metal ions is used. Thus the chelator is capable of complexing with metal ions so as to reduce their ability to adversely affect the solubility or dispersibility of the solid composition. The chelator may be a phosphate. It may be an organic phosphate, in particular an organic polyphosphate. A suitable chelator is sodium phytate. In some instances more than one chelator may be present. Each may be as described above.

The chelator may be present in an amount effective to ensure that the composition is dilutable and/or soluble in hard water. It may be present at about 1 to about 5% by weight, or about 1 to 3, 3 to 5 or 2 to 4%, e.g. about 1, 2, 3, 4 or 5%. In some instances, for example for use with particularly hard or metal rich water, it may be present at greater than 5%. In the event that more than one chelator is present, the combined amounts of the chelators may be as described above.

The composition also includes a preservative. The function of the preservative is to prevent or inhibit microbial and/or fungal infestation of the composition, i.e. to improve its storage life. The preservative may be an antifungal agent or may be an antibacterial agent or may be a general antimicrobial agent. More than one preservative may be used. For example there may be an antifungal agent and an antimicrobial agent. A commonly used preservative is sodium benzoate, which has both antibacterial and antifungal activity. It will be understood that the sodium benzoate may be at least partially present as benzoic acid, depending on the pH of the composition or its diluted form. Another suitable preservative is dehydroacetic acid, which also has both antibacterial and antifungal activity. Depending on the pH, this may be at least partially present as its salt. Where reference is made here to “antibacterial activity” this may refer to bactericidal activity (i.e. killing bacteria) and/or to bacteriostatic activity (i.e. preventing bacteria from multiplying). Similarly antifungal activity may refer to fungicidal and/or fungistatic activity.

The preservative may be present in an amount effective to provide a desired shelf life. The desired shelf life may be at least 12 months, or at least 15, 18, 21, 24, 30 or 36 months. The preservative may be present at about 5 to about 20%, or about 5 to 10, 10 to 20, 10 to 15 or 7 to 12%, e.g. about 5, 6, 7, 8, 9, 10, 11, 12, 13, 14, 15, 16, 17, 18, 19 or 20%. In the event of more than one preservative being present, the combined amount may be as set out above. A suitable combination of preservatives is sodium phytate and dehydroacetic acid. They may be present in a ratio of from 1:2 to 2:1 on a weigh basis, or from about 1:2 to 1:1, 1:1 to 2:1, 2:3 to 3:2, 2:3 to 1:1 or 1:1 to 3:2, e.g. about 1:2, 2:3, 1:1, 3:2 or 2:1.

For the preservative to be effective, the pH of the composition, or of its diluted form, should be mildly acidic. A suitable pH is from about 5 to about 6.5. It may be from about 5 to about 5.5, 5.5 to 6 or 6 to 6.5, e.g. about 5, 5.2, 5.4, 5.6, 5.8, 6, 6.2, 6.4 or 6.5. Suitable buffers are well known to those skilled in the art. A suitable example is a citrate buffer. The citrate may be in the form of its sodium salt or its potassium salt. By varying the ratio of citrate to citric acid, a desired pH in the above range may be achieved. It should be noted that other components in the composition may also have buffering effects, for example benzoic acid/benzoate, used as a preservative, and phytic acid/sodium phytate, used as a chelator, may affect the pH of the composition or its diluted form. Citric acid/citrate buffer may be a suitable buffer, since the citric acid may also act as a water softener, complexing with water hardness so as to complement the activity of the chelator described above.

The pH buffer may be present at a total concentration (i.e. acid and conjugate base) of about 5 to about 10% w/w, or about 5 to 8, 5 to 6, 6 to 10, 8 to 10 or 7 to 8%, e.g. about 5, 6, 7, 7.5, 8, 9 or 10% w/w. The ratio of acid to conjugate base may be from about 1:5 to about 5:1 on a weight basis, or about 1:4 to 4:1, 1:3 to 3:1, 1:2 to 2:1 1:4 to 1:1, 1:2 to 1:1, 1:1 to 1:4 or 1:1 to 1:2, e.g. about 1:5, 1:4, 1:3, 1:2, 2:3, 1:1, 3:2, 2:1, 3:1, 4:1 or 5:1.

In order for the composition to remain as a free-flowing powder, the composition may comprise an anticaking agent. Suitable anticaking agents include alkaline earth salts of long chain fatty acids, e.g. magnesium stearate and calcium stearate, silica, e.g. hydrated silica, aluminosilicates e.g. sodium aluminosilicate, diatomaceous earth, talc and starch. More than one anticaking agent may be used, or a single anticaking agent may be used.

Anticaking agents may serve to adsorb and/or absorb (i.e. sorb) liquids which might otherwise adhere particles of the composition together. Such adhering may cause caking and/or stickiness. It may cause a reduction in the free-flowing nature of a powder composition. Liquids that may be present and which may be sorbed by the anticaking agent include water (e.g from the atmosphere or from one or more added ingredients), a fragrance or essential oil, compatibilisers used with the fragrance or essential oil, emollients etc. Thus the anticaking agent may be viewed as a carrier for liquids in the formulation that may otherwise adversely affect the physical properties of the composition.

The anticaking agent, or the combination of anticaking agents, may be present from about 1 to about 5% w/w, or from about 1 to 3, 3 to 5 or 2 to 4%, e.g. about 1, 2, 3, 4 or 5%.

An emollient and/or moisturiser may also be included in the composition. This serves to make the diluted product kinder to the skin. These materials are commonly long chain fatty acid esters, alcohols or acids. They may be for example C8, C10, C12, C14, C16 or C18 fatty acid esters, alcohols or acids, for example cetyl alcohol, isopropyl myristate, isopropyl palmitate, glyceryl caprylate or stearyl alcohol. Combinations of emollients may be used.

The emollient, or the combination of emollients, may be present from about 1 to about 5% w/w, or from about 1 to 3, 3 to 5 or 2 to 4%, e.g. about 1, 2, 3, 4 or 5%.

A further component of the composition may be a solid skin conditioner. This may additionally function as a filler. Suitable solid skin conditioners include sugars/polyols, for example erythritol, pentaerythritol or xylitol. This may be present at up to about 25% of the composition by weight, or up to about 20, 15 or 10%, or from about 1 to about 25%, or about 15 to 25, 20 to 25, 10 to 20 or 15 to 20%, e.g. about 10, 15, 20 or 25%.

In order to make the composition more attractive to consumers, it may be advantageous to include a fragrance and/or essential oil. Suitable such materials include lavender oil, frankincense oil, peppermint oil, rosemary oil, sweet orange oil and combinations of these, or combinations of these with other fragrant oils such as bergamot, patchouli, sandalwood, ylang ylang, geranium etc. In general these need only be present in small amounts, commonly about 0.1 to about 0.5% by weight of the composition, or about 0.1 to 0.3, 0.2 to 0.5 or 0.3 to 0.4%, e.g. about 0.1, 0.2, 0.3, 0.4 or 0.5%. It should preferably be present in sufficient quantity to provide the diluted composition with a suitably strong fragrance but not sufficient to reduce the free-flowing nature of the composition in its powdered form.

As the fragrances/essential oils used in the composition are typically not water soluble, it may be useful to include a compatibiliser in order to combatibilise the fragrance/essential oil with the aqueous environment of the diluted composition. The compatibiliser may be a solubiliser. It may be an emulsifier. It may serve to render the fragrance/essential oil compatible with, optionally soluble or emulsifiable in, an aqueous environment. Suitable compatibilisers include surfactants, a wide range of which are well known to the skilled person. A suitable compatibiliser is cocoyl proline, which is also known for its hydrating properties.

Commonly the compatibiliser is present in the composition at about 5 to about 10 times the amount of the fragrance/essential oil, or about 5 to 8, 7 to 10 or 6 to 8 times, e.g. about 5, 6, 7, 8, 9 or 10 times the amount. It may be present in the composition at about 0.5 to about 5% w/w, or about 0.5 to 2, 1 to 5, 2 to 5 or 2 to 3%, e.g. about 0.5, 1, 1.5, 2, 2.5, 3, 3.5, 4, 4.5 or 5%.

The composition of the present invention may be a free-flowing powder. In this context, a powder may be considered to be a particulate substance of small particle size. The maximum, or mean, particle diameter may be about 2mm, or about 1.5, 1 or 0.5mm When stored in a sealed container with less than 25% by volume headspace, the composition may resist particle aggregation for at least 1 month, or at least 2, 3, 4, 5, 6, 12, 18 or 24 months.

The composition is commonly a low water composition. The maximum water content of the composition may be about 2%, or may be about 1.5, 1, 0.5, 0.2 or 0.1%.

The invention also encompasses a process for making a liquid foaming composition, for example a liquid foaming hand soap. This process comprises dispersion and/or dissolving the solid foaming composition described above in water. It will be understood that the water used in this process may be pure or may have dissolved and/or dispersed components therein. Commonly the water will be at least about 95% pure on a w/v basis, or at least about 96, 97, 98, 99, 99.5 or 99.9% pure on a w/v basis. It will also be recognised that not all of the components of the solid foaming composition are soluble in water. For example, hydrated silica, which may be used as an anticaking agent may not dissolve and hence may be dispersed in the water. Similarly essential oils which may be present as fragrances may not be water soluble at the level at which they are present, and may be dispersed in the water, commonly as emulsion particles. The emulsification of such materials may be facilitated by emulsifiers/surfactants present in the composition, for example the compatibiliser, the solid surfactant and others.

The dissolving/dispersing may be at a ratio of water to solid foaming composition of about 20 to about 50 parts to 1 part of the solid foaming composition on a w/w basis, or about 20 to 40, 20 to 30, 30 to 50, 40 to 50 or 30 to 40 parts to 1 part of the solid foaming composition, e.g. about 20, 25, 30, 35, 40, 45 or 50 parts to 1 part of the solid foaming composition. Higher dilutions may be used as well if a less powerful cleanser is desired. Alternatively lower dilutions may be used if a more powerful cleanser is desired or if a concentrate is to be prepared for subsequent dilution to the actual liquid foaming composition.

The water used in dissolving or dispersing the solid foaming composition may be at a slightly elevated temperature. It may be from about 30 to about 90° C., or about 30 to 70, 30 to 50, 50 to 90 or 50 to 70° C., e.g. about 30, 40, 50, 60, 70, 80 or 90° C. It may alternatively be at ambient temperature, e.g. about 20 or 25° C. Once the water and the solid foaming composition have been combined, they may be agitated so as to facilitate dissolution and/or dispersion. They may be agitated for at least about 10 seconds or at least about 20 or 30 seconds, or for about 10, 15, 20, 25, 30, 35, 40, 45, 50, 55 or 60 seconds. The agitation may comprise stirring, shaking, swirling or any combination of these.

The resulting liquid foaming composition may be used as a liquid soap, for example a body wash or a hand wash. It may be substantially homogeneous. It may remain substantially homogeneous on standing for at least 1 day, or at least 1 week or at least one month. The term “substantially homogeneous” should be taken to mean that the amount of any particular component in the upper half of the liquid foaming composition is not more than 10% greater or less than the amount of that component in the lower half thereof.

This liquid foaming composition may be resistant to microbial or fungal infestation for at least 1 day, or at least 1 week or at least one month. It may be biodegradable. It may be palm oil free. It may be non-toxic to humans.

The present invention also encompasses a process for making the solid foaming composition described above.

In its simplest form, the process may comprise combining all of the components as discussed earlier and blending them. A convenient form of blending is a ribbon blender, although other means for blending may be used, for example a planetary mixer, a stirrer, a shaker etc. Any of these may be used for all mixing or blending steps discussed below other than those specified as using a mixer granulator.

More commonly, the process involves initially comminuting, e g milling, the solid surfactant to reduce its particle size. It may then comprise sieving the milled solid surfactant. This may be through a sieve with holes of less than about 5 mm, or less than 4, 3 or 2 mm, e g holes of about 2 mm, 3 mm, 4 mm or 5 mm. It may be through a 4-12 mesh sieve, e.g. 4, 5, 6, 7, 8, 9, 10, 11 or 12 mesh sieve. A 10 mesh sieve (2 mm) is suitable. Sieving ensures that large particles are excluded from the composition.

The chelator, the preservative and the pH buffer and, if used, the solid skin conditioner, are then combined with the sieved solid surfactant. They may be premixed prior to being combined with the surfactant. After the premixing, or after combining with the surfactant they may be again sieved. They may be sieved through a fine sieve. It may have holes less than about 2 mm, or less than 1 mm. The sieve may be a 10-35 mesh sieve. It may be a 10, 12, 15, 16, 18, 20, 25, 30 or 35 mesh sieve.

If used, the essential oil and the compatibiliser may be premixed before combining with other components. They may be mixed for sufficient time to form a clear mixture. This mixture is commonly a liquid, however is present in the final composition in sufficiently low quantity as to not jeopardise the solid properties thereof.

The mixed essential oil and compatibiliser may then be combined with at least a portion of the anticaking agent. Commonly it will be combined with approximately half of the total amount of anticaking agent. It may be combined with anywhere between about 20% and 100% of the anticaking agent, for example about 20 to 80, 20 to 60, 20 to 50, 30 to 100, 50 to 100 or 50 to 80%, e.g. about 20, 30, 40, 50, 60, 70, 80, 90 or 100% of the anticaking agent. The combining of the essential oil/compatibiliser mixture with the anticaking agent may comprise adding the essential oil/compatibiliser mixture to the anticaking agent while mixing the anticaking agent. Thus the anticaking agent may be in a mixer-granulator or other suitable device, and the essential oil/compatibiliser mixture added thereto. It is commonly added slowly over a period of time. The rate of addition is preferably sufficiently slow as to allow incorporation of the essential oil/compatibiliser mixture into the anticaking agent without undue aggregation. The time, and rate, of addition may depend on the rate of mixing, on the volume of each of the components, on the temperature at which the addition occurs and on other factors. The skilled person will be able to determine a suitable rate of addition by routine trial and error. The speed of the mixer granulator may be increased during and/or after the addition of the essential oil/compatibiliser mixture into the anticaking agent. This can serve to combine the ingredients effectively and ensure that the particle size is kept small. In the event that the fragrance/essential oil is not used, then the compatibiliser will also not be added. In this event, this addition of the anticaking agent may be omitted although later addition, as discussed below, will be performed.

In a mixer granulator, granules are formed by the rising, whirling and tumbling motion of the material. The mixing of the ingredients is achieved by rotation of an impeller and a chopper, commonly at high speed. This may impart high shear to the contents of the mixer granulator.

The resulting granular material (which comprises the essential oil, the compatibiliser and the anticaking agent) may then be combined with the previously prepared mixture which (which comprises the solid surfactant, optionally the solid skin conditioner, the chelator, preservative and pH buffer). In the event that the essential oil and compatibiliser are not used, the anticaking agent may be added directly to the previously prepared mixture without subjecting it to the mixer granulator. This combining may be performed in a suitable mixing device, for example a ribbon blender or planetary mixer.

The emollient may be added directly to the resultant mixture. However it is convenient to premix it with a portion of the anticaking mixture. As some suitable emollients are liquids, adding them directly to the above mixture can result in uneven mixing through the final composition. Combining the emollient with anticaking agent (which is a solid) can provide a granular or powder material which can readily be dispersed evenly through the composition. If the emollient is a low melting solid (for example, glyceryl caprylate has a melting point of about 34° C.), it may be preheated to melt it and then added to the anticaking agent. This may be the balance of the anticaking agent which, in some embodiments was earlier combined with the essential oil and compatibiliser. It should be noted that, in the event that no essential oil or compatibiliser is used, then the earlier portion of anticaking agent (which was added to earlier mixture) may be omitted and 100% of the anticaking agent added with the emollient.

The emollient and anticaking agent may be combined as described earlier for combining the essential oil/compatibiliser with the anticaking agent. Thus the combining may be conducted in a mixer granulator, optionally with increasing speed so as to generate a finely divided mixture.

The combined emollient and anticaking agent may then be combined with the earlier mixture and mixed for sufficient time for a uniform mixture to be achieved. This uniform mixture is the final solid foaming composition of the invention.

### EXAMPLE

| | | | | | |
|---|---|---|---|---|---|
| A | Iselux (Flakes) | Sodium Lauroyl | 53.8000 | AS Harrison | Innospec |
| | | Methyl Isethionate | | | |
| B | Erythritol | Erythritol | 20.0000 | Brenntag | Jungbunzlauer |
| B | Dermofeel PA-12 | Sodium Phytate | 2.5000 | Avenir* | Dr Straetmans |
| B | Microcare NB | Sodium Benzoate | 5.0000 | Thor | Thor |
| | (Ecocert) | | | | |
| B | Citric Acid | Citric Acid Water | 2.5000 | Manufacture | Manufacturer |
| | (Monohydrate) | | | | |
| B | Sodium Citrate | Sodium Citrate | 5.0000 | Manufacturer | Manufacturer |
| B | Microcare DHA | Dehydroacetic Acid | 5.0000 | Thor | Thor |
| C | Lavender | Lavandula | 0.2000 | New | New Directions |
| | Population Essential | Angustifolia | | Directions | |
| | Oil | (Lavender) Flower Oil | | | |
| C | Natisol | Cocoyl Proline | 2.0000 | Connell* | Sinerga |
| D | Sipemat 22S | Hydrated Silica | 1.0000 | Chemiplas | Evonik |
| E | Lexgard GMCY | Glyceryl Caprylate | 2.0000 | AS Harrison* | Inolex |
| F | Sipemat 22S | Hydrated Silica | 1.0000 | Chemiplas | Evonik |

*Ingredients may be sourced from on-line retailers inc. New Directions, Trulux, Aussie Soap Supplies, Escentials of Australia etc.
Process

| | |
|---|---|
| 1 | Wear appropriate PPE when handling powders, including suitable respiratory masks, |
| | safety glasses and gloves. |
| 2 | Pass Phase A through a mill fitted with a ~2 mm (10 mesh) screen and into a ribbon |
| | blender. |
| 3 | Combine Phase B ingredients into the ribbon blender, mix until uniform, then pass |
| | through a mill fitted with a ~1 mm (18 mesh) screen and back into the ribbon blender. |
| 4 | Premix Phase C in a suitable vessel, mix until clear. |
| 5 | Transfer Phase D into a mixer granulator and begin mixing, then SLOWLY add |
| | Phase C premix, increase mixing speed and use chopper to combine. |
| 6 | Transfer Phase C/D premix into the ribbon blender. |
| 7 | Transfer Phase F into the mixer granulator and begin mixing, then SLOWLY add |
| | Phase E (premelted), increase mixing speed and use chopper to combine. |
| 8 | Transfer Phase E/F premix into the ribbon blender, mix until uniform. |

| Description | Limits | Method |
|---|---|---|
| Appearance | Fine powder - To match standard | QC-005 |
| Colour | White to off-white - To match standard | QC-005 |
| Odour | Lavender, characteristic - To match standard | QC-005 |
| pH @ 25° C. | 5.0-5.5 (5% soln.) | QC-001 |
| SG @ 25° C. | N/A | QC-003 |
| Viscosity @25° C. | N/A | QC-002 |
| Total Aerobic Microbial | ≤100 | BP - External |
| Count (CFU/g) | | |
| Total Yeasts & Moulds | ≤10 | BP - External |
| (CFU/g) | | |
| Pseudomonas Aeruginosa/g | Not detected in 1 g | BP - External |
| Staphylococcus Aereus/g | Not detected in 1 g | BP - External |

Ingredient List
Sodium Lauroyl Methyl Isethionate, Erythritol, Dehydroacetic Acid, Sodium Citrate, Sodium
Benzoate, Sodium Phytate, Citric Acid, Glyceryl Caprylate, Hydrated Silica, Cocoyl Proline,
[FOLLOWING INGREDIENTS IN ANY ORDER] Water, Lavandula Angustifolia
(Lavender) Flower Oil

### Industrial Application

The solid composition may be combined with water so as to produce a foaming liquid hand wash, body wash or similar product.

### Interpretation

### Markush Groups

Where features or aspects of the invention are described in terms of Markush groups, those skilled in the art will recognise that the invention is also thereby described in terms of any individual member or subgroup of members of the Markush group.

### Chronological Sequence

For the purpose of this specification, where method steps are described in sequence, the sequence does not necessarily mean that the steps are to be carried out in chronological order in that sequence, unless there is no other logical manner of interpreting the sequence or unless that specific order is indicated.

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

As used herein the term “and/or” means “and” or “or”, or both.

As used herein “(s)” following a noun means the plural and/or singular forms of the noun.

As used herein, the term “may” encompasses both the stated proposition and its opposite. Thus, for example, the phrase “A may be B” envisages both “A is B” and “A is not B” unless the specific context indicates otherwise.

### Comprising, Including, Consisting Essential of

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

The term “consisting essential of”, or variations thereof, is used to indicate that the stated integers are the only ones intentionally added and that other integers which may be present to do not substantially affect the working of the invention. Thus for example, “A consists essentially of B and C” requires that B and C be present and that no further components are added intentionally. It does not preclude the presence in A of, for example, impurities present in B and/or C, or entrained air incorporated during mixing of B and C.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the compositions described are applicable to the personal care industry.

1. A solid foaming composition comprising:
a solid surfactant;
a chelator;
a preservative;
a pH buffer;
an anticaking agent; and
an emollient.

2. The composition of claim 1 wherein the surfactant is present at about 50 to about 90% of the composition.

3. The composition of claim 1 wherein the chelator is present at about 1 to about 5% of the composition.

4. The composition of claim 1 wherein the preservative is present at about 5 to about 20% of the composition.

5. The composition of claim 1 wherein the pH buffer is present at about 5 to about 10% of the composition.

6. The composition of claim 1 wherein the anticaking agent is present at about 1 to about 5% of the composition.

7. The composition of claim 1 wherein the emollient is present at about 1 to about 5% of the composition.

8. The composition of claim 1 additionally comprising a solid skin conditioner.

9. The composition of claim 8 wherein the skin conditioner is present at about 10 to about 25% of the composition.

10. The composition of claim 1 additionally comprising an essential oil and a compatibiliser.

11. The composition of claim 10 wherein the essential oil is present at about 0.1 to about 0.5% of the composition and the compatibiliser is present at from 5 to about 10 times the weight of the essential oil.

12. The composition of claim 1 comprising less than about 0.5% free water.

13. A process for making a solid foaming composition comprising blending a solid surfactant, a chelator, a preservative, a pH buffer, an anticaking agent and an emollient.

14. The process of claim 13 comprising milling the solid surfactant prior to said blending.

15. The process of claim 13 comprising:
Step A: combining the chelator, the buffer and the preservative to produce a first blend and
Step B: blending the first blend with the solid surfactant to form a second blend.

16. The process of claim 15 comprising:
Step C: combining the second blend with at least a portion of the anticaking agent to form a third blend.

17. The process of claim 16 wherein Step C comprises:
combining an essential oil and a compatibiliser to form an oil-compatibiliser mixture and
adding the oil-compatibiliser mixture to the anticaking agent under agitation, so as to form a fourth blend, and
combining the second blend and the fourth blend so as to form the third blend.

18. The process of claim 16 comprising:
Step D: combining the emollient with the third blend so as to form the composition.

19. The process of claim 18 wherein Step D comprises:
combining the emollient with a portion of the anticaking agent to form a fifth blend; and
adding the fifth blend to the third blend so as to form the composition.

20. The process of claim 15 wherein step A comprises:
combining the chelator, the buffer, the preservative and a solid skin conditioner so as to produce the first blend.

21. A process for making a liquid foaming composition, said process comprising:
combining the composition of claim 1 with water in a ratio of 1 part by weight of said composition to from 20 to 50 parts by weight of water, said water being at a temperature of between about 30 and about 90° C.; and
agitating the resulting mixture for a time of between about 10 and 60 seconds.

22. (canceled)
